(19) United States
(12) Patent Application Publication
(10) Pub. No.: US 2021/0298589 A1
(43) Pub. Date: Sep. 30, 2021

(54) SYSTEMS AND METHODS FOR EXAMINING AND TREATING INTRAPELVIC CONDITIONS

(71) Applicant: The Brigham and Women's Hospital, Inc., Boston, MA (US)

(72) Inventor: Jon I Einarsson, Boston, MA (US)

(21) Appl. No.: 17/345,742

(22) Filed: Jun. 11, 2021

Related U.S. Application Data
(63) Continuation of application No. PCT/US2019/065723, filed on Dec. 11, 2019.
(60) Provisional application No. 62/778,102, filed on Dec. 11, 2018.
(60) Provisional application No. 62/887,053, filed on Aug. 15, 2019.

(51) Int. Cl. A61B 1/303; A61B 1/015; A61B 10/04; A61B 10/02

(52) U.S. Cl. CPC A61B 1/303; A61B 1/015; A61B 2010/045; A61B 10/0291; A61B 10/04

(57) ABSTRACT

An illustrative method in accordance with the disclosure includes introducing a visualization scope having a proximal end and a distal end into a patient's uterus by way of the vagina and cervix, advancing the distal end of the visualization scope into one of the fallopian tubes from the uterus, and advancing the distal end of the visualization scope out of said fallopian tube into an abdominal cavity of the patient.

### CROSS-REFERENCE TO RELATED APPLICATIONS

This patent application claims the benefit of priority to and is a continuation of International Patent Application No. PCT/US19/65723, filed Dec. 11, 2019, which claims the benefit of priority to U.S. Provisional Patent Application No. 62/887,053, filed Aug. 15, 2019, and U.S. Provisional Patent Application No. 62/778,102, filed Dec. 11, 2018. The contents of each of the foregoing patent applications is incorporated by reference herein in its entirety for all purposes.

### BACKGROUND

Endometriosis is a common condition with approximately 10% of women affected by this disease. Diagnostic delay is common with the average delay being approximately 7 years. There is currently no reliable diagnostic modality since most endometriosis lesions are not visible on traditional imaging. Therefore, definitive diagnosis of endometriosis currently requires laparoscopic surgery under general anesthesia in the operating room. Ovarian cancer is a rare, but deadly disease that affects approximately 1.5% of women. There are no effective screening methods available in high risk women and they are generally offered surgical removal. Better screening methods are urgently needed. The present disclosure provides solutions for these and other problems and set forth hereinbelow.

### SUMMARY OF THE DISCLOSURE

The purpose and advantages of embodiments of the present disclosure will be set forth in and become apparent from the description that follows. Additional advantages of embodiments of the present disclosure will be realized and attained by the methods and systems particularly pointed out in the written description and claims hereof, as well as from the appended drawings.

To achieve these and other advantages and in accordance with the purpose of the disclosure, as embodied herein, in accordance with one aspect, the disclosure provides methods for evaluating one or more intrapelvic conditions.

An illustrative method in accordance with the disclosure includes introducing a visualization scope having a proximal end and a distal end into a patient's uterus by way of the vagina and cervix, advancing the distal end of the visualization scope into one of the fallopian tubes from the uterus, and advancing the distal end of the visualization scope out of said fallopian tube into an abdominal cavity of the patient.

In various embodiments, the visualization scope can be directed from outside the patient through the vagina, uterus, and fallopian tube into the abdominal cavity without puncturing a tissue structure, although the present disclosure does not exclude puncturing tissue structures.

If desired, the method can further include directing liquid through the visualization scope when the distal end of the visualization scope is in said fallopian tube to distend said fallopian tube. This can facilitate examining at least one structure in said distended fallopian tube for an abnormality. The method can still further include advancing the distal end of the visualization scope into a pelvic region of the abdominal cavity. If desired, the method can further include directing liquid through the visualization scope when the distal end of the visualization scope is in the pelvic region of said abdominal cavity. In some implementations, the method can further include examining at least one anatomical structure within the pelvic cavity for at least one abnormality. For example, the anatomical structure can include one or more of (i) a surface of the patient's uterus, (ii) at least one of the patient's ovaries, and (iii) the patient's bowels. In some embodiments, the visualization scope can be used to detect at least one abnormality associated with endometriosis.

If desired, embodiments of the disclosed visualization scope can be used as a screening tool to detect at least one abnormality associated with cancer, and the method can include examining one or more of (i) a surface of the patient's uterus, (ii) at least one of the patient's ovaries, (iii) the patient's bowels, (iv) at least one of the patient's fallopian tubes, and (v) the patient's peritoneum.

In some embodiments, the method can include aspirating a fluid sample from said abdominal cavity, and performing at least one testing procedure on the fluid sample in order to detect at least one abnormality. The at least one testing procedure can be configured to detect at least one of (i) cancerous tissue, and (ii) endometriosis, for example.

If desired, the method can further include inserting at least one biopsy tool into the abdominal cavity to take at least one sample of said at least one anatomical structure, wherein the at least one biopsy tool can be inserted into the abdominal cavity via (i) a channel of the visualization scope, (ii) by utilizing the visualization scope as a rail, or (iii) through the patient's other fallopian tube. If desired, the visualization scope can include an electronic photodetector, such as a photodetector chip, disposed proximate to the distal end of the visualization scope for receiving incoming light. Alternatively, the visualization scope can include a fiber optic element for transmitting a signal of incoming light to a photodetector elsewhere in the scope or to a device connected to the scope. If desired, the visualization scope can include light emitting device proximate to the distal end of the visualization scope, such as a LED or a lens connected to a light conductor, such as a fiber optic light conductor. The method can include directing signals from the photodetector to a processor.

In some embodiments, the method can further include directing signals from the processor to a display screen. The method can also include tilting the pelvis of the patient in order to move the bowels out of the way of the visualization scope. If desired, the method can include directing liquid into the pelvic cavity to facilitate movement of the bowels.

In some embodiments, the method can include directing a laser light signal through the visualization scope to treat tissue inside of the patient, such as by irradiating the tissue with laser light. In some embodiments, the diagnostic method can be repeated a plurality times over a plurality of examinations in order to track progress of a treatment regimen of the patient.

The disclosure provides various embodiments of a scope for examining an intrapelvic condition. In one illustrative embodiment, a scope for evaluating an intrapelvic condition is provided that can include a handle having a proximal end and a distal end, a tubular body extending from the distal end of the handle, the tubular body including a first scope operably associated therewith, wherein the tubular body further defines a channel along its length, and a second scope slidably disposed in the lumen of the tubular body, the second scope being configured and arranged for being advanced along a fallopian tube of a patient.

In some embodiments, the second scope has a steerable distal end. If desired, the second scope can define a lumen along its length. The first and second scopes are preferably fluid resistant. In some embodiments, the second scope can be configured to traverse a first fallopian tube of a patient to gain access to the pelvic cavity to perform at least one testing procedure on at least one intrapelvic anatomical structure. The scope can further include at least one biopsy tool. If desired, the scope can further include at least one lumen for directing fluid therethrough. Moreover, the scope can further include one or more light conduit(s) for directing light therethrough, such as light for illumination, or light for therapeutic application (e.g., laser light). In various embodiments, the first and/or second scopes includes a visualization element at a distal end thereof. Preferably, the second scope includes an atraumatic lens disposed over the visualization element to prevent trauma to the Fallopian tubes and other structures.

In further accordance with the disclosure, the inner, or second, scope can have a diameter of about 5 French. The outer, or first, scope can be a standard hysteroscope defining a 5 Fr passage therethrough for receiving the second scope. If desired, the lumen of the second scope can include a radial stiffening element configured to help prevent the lumen of the second scope from collapsing radially inwardly when under negative fluid pressure. If desired, the second scope can include an uneven outer surface configured to collect a tissue sample from a patient as the second scope passes over tissue of a patient. For example, the uneven outer surface can include a plurality of hair-like elements. If desired, the uneven outer surface can be formed into an outer surface of the second scope. The uneven outer surface can be configured to collect a tissue sample and enhance hoop stress resistance of the second scope while maintaining blending flexibility. The device can further include a pressurized fluid source coupled to the lumen of the second scope. The pressurized fluid source can include a motorized fluid pump or a syringe, for example. The pressurized fluid source can include a mechanical lock for maintaining an applied pressure.

The disclosure further provides methods for evaluating an intrapelvic condition, including introducing a visualization scope having a proximal end and a distal end into a patient's cul de sac, and performing at least one of a diagnostic or therapeutic procedure inside the patient's cul de sac. The method can further include introducing a needle through the vagina and into the cul de sac to define a passageway through which the visualization scope can pass. The diagnostic procedure can include at least one of aspirating fluid and obtaining a tissue sample. The therapeutic procedure can include delivering a beneficial agent to tissue in the cul de sac, among other things.

It is to be understood that both the foregoing general description and the following detailed description are exemplary and are intended to provide further explanation of the claimed embodiments.

The accompanying drawings, which are incorporated in and constitute part of this specification, are included to illustrate and provide a further understanding of the methods and systems of the disclosure. Together with the description, the drawings serve to explain the principles of embodiments of the present disclosure.

### BRIEF DESCRIPTION OF THE DRAWINGS

FIGS. 1A-1B are illustrations of an example of a scope in accordance with the present disclosure.

FIGS. 2-4 are illustrations of an example of a method in accordance with the present disclosure.

FIG. 5 is an illustration of a distal portion of an implementation of a scope in accordance with the present disclosure.

FIGS. 6A-6E illustrate aspects of a further implementation in accordance with the present disclosure.

### DETAILED DESCRIPTION OF THE PREFERRED EMBODIMENTS

Reference will now be made in detail to the present preferred embodiments of the disclosure, examples of which are illustrated in the accompanying drawings. The methods and corresponding steps of the disclosure will be described in conjunction with the detailed description of the system.

The devices and methods presented herein may be used for myriad purposes. Generally, the devices described herein may be used for gynecological examination purposes. But, the devices disclosed herein can similarly be used for providing diagnostic tools for examining respiratory structures such as lung bronchi and bronchioles, as well as cranial passages such as sinus passages and related structures, for example.

In accordance with one aspect of the disclosure, a scope is provided for diagnosing and/or treating an intrapelvic condition.

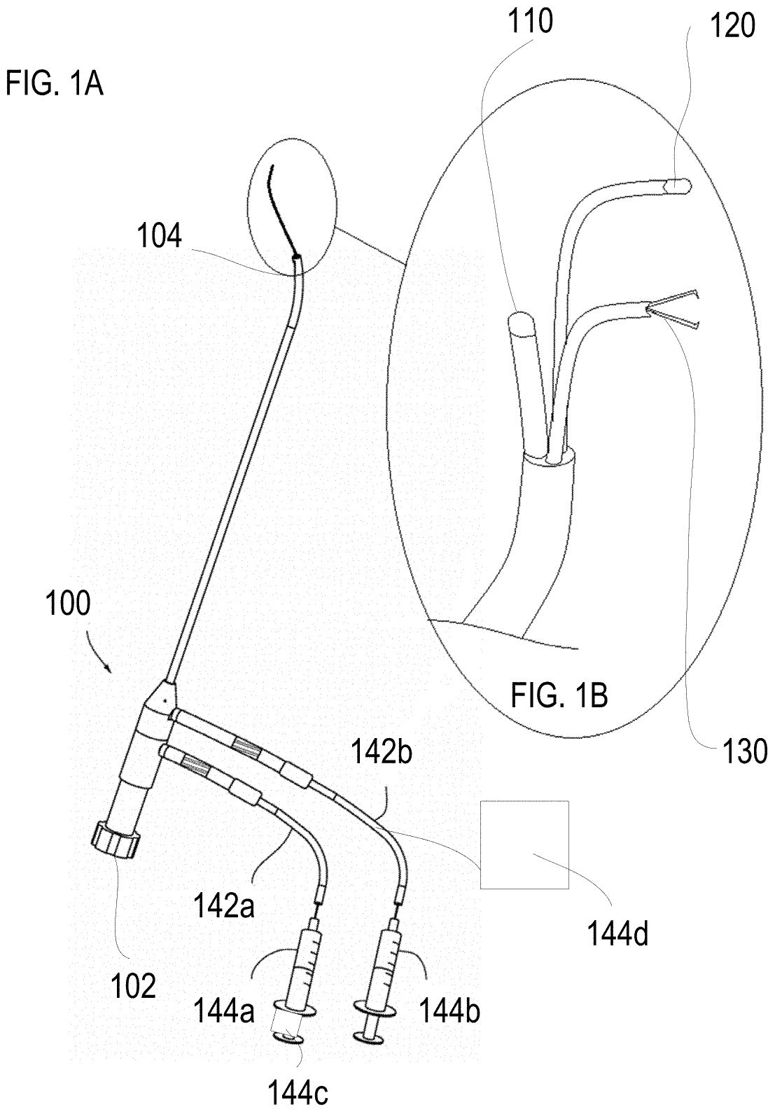

For purpose of explanation and illustration, and not limitation, a partial view of an illustrative embodiment of the scope 100 in accordance with the disclosure is shown in FIGS. 1A-1B.

As illustrated, scope 100 has proximal end 102 and distal end 104, and a tubular body extending from the distal end of a handle. The tubular body includes a first scope 110 operably associated therewith. As illustrated, the tubular body further defines two additional channels along its length, and includes a second scope 120 that is slidably disposed in the lumen of the tubular body. As discussed below, the second scope being configured and arranged for being advanced along a fallopian tube of a patient. A tool 130, that may be, for example, a biopsy tool, a further visualization tool, a laser catheter, or the like, can also be slidably disposed within a further lumen of the tubular body of the scope 100. If desired, the scope 120 can be a standalone device that, if desired, can be used to deliver or collect fluids from a target location inside of a patient.

The scope 100 can be (e.g., a 5 French) flexible catheter that can be provided with one or more digital light capturing devices, such as a CMOS chip (or lens and fiber optic conductor) and a light source (e.g., fiber optic or LED) at its distal tip. The CMOS can have a width, for example, of about 0.6 millimeters or greater, in any increment of 0.1 millimeters. The circuitry coupled to the CMOS chip can be coupled to electronics to convert received light signals into an image. The circuitry can be coupled to a monitor to permit real time visualization of the image received by the CMOS chip.

The scope 100 can also defines a channel along its length for fluid to be pushed and/or aspirated through the channel in addition to those illustrate in FIG. 1, or in place, for example, of device 130. For example, instead of device 130, a different aspiration catheter can be used to direct fluid into or remove a fluid sample from the abdominal or pelvic cavity. The distal end 102 of the scope 100 preferably has a flexible distal end to permit the tip to be controllably articulated. Alternatively, the distal end can be bent into a predetermined angle (e.g., any desired angle between about 5 degrees and about 45 degrees, in increments of about one degree, such as about 5, 6, 7, degrees up to about 45 degrees). The tip is preferably movable, such as by rotation, from outside the patient to obtain different views of the pelvis.

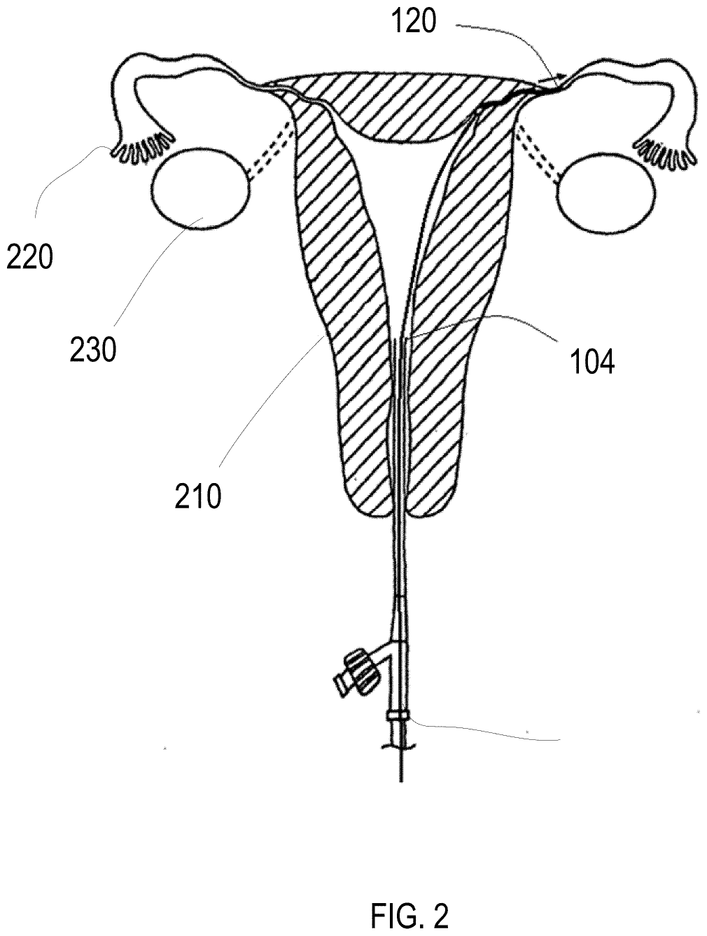

With reference to FIGS. 2-4, in one implementation of use of the scope 100, a patient can come into the office for an in-office hysteroscopy. With reference to FIG. 2, the office hysteroscopy proceeds allowing the surgeon to examine the uterine cavity by directing the distal end 104 of the scope 100 through the vagina and cervix into the uterus 210. FIG. 2 depicts the uterus 210, ovaries 230 and fallopian tubes 220. However, the surgeon can then advance the second scope 120 (e.g., size of 5 French) through the scope 100 and steer it into one of the patient's fallopian tubes 220 via its respective comu. Scope 120 can itself be provided with a small irrigation channel that can be fed by a fluid source that is actuated by a plunger, syringe or the like (e.g., 144a). By way of further example, the plunger can include a compression spring and/or a lockout 144c (e.g., surrounding the plunger shaft) that is compressed when the plunger is depressed, and causes the plunger to aspirate fluid and/or cells out of a patient when the plunger is released. A lockout can, for example, lock the plunger in place when it is compressed to permit time to pass between the introduction of fluid into the peritoneal cavity and collection of the fluid sample. By way of a further example, the fluid source can include a mechanical or electrically powered pump 144d (e.g. battery powered or plug-in) such as a peristaltic pump, diaphragm pump and the like. The pump 144d can be operated by a computer processor 144e that can regulate the applied pressure, flow rate and timing of fluid introduction and extraction.

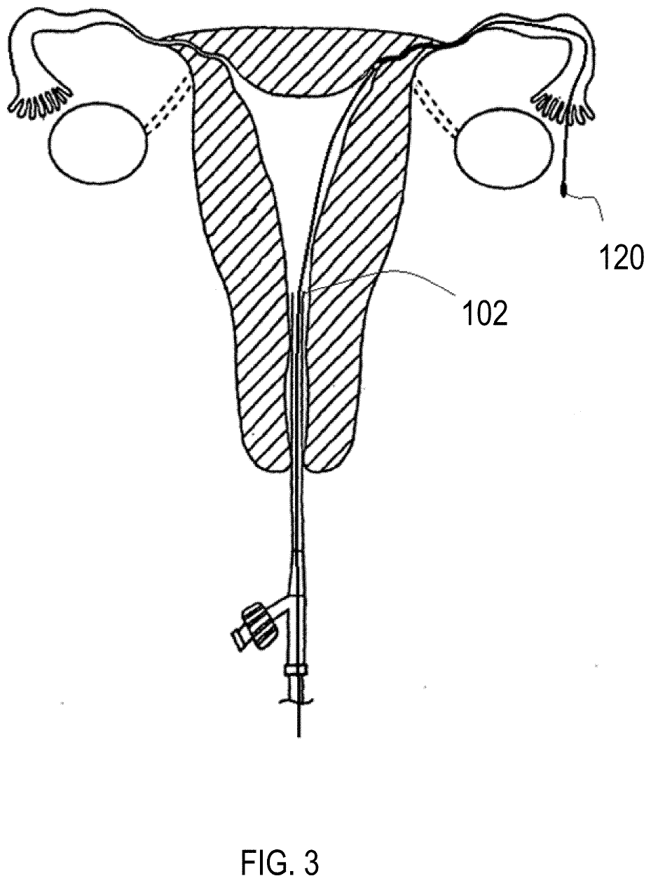

Regardless as to how the fluid is introduced, fluid, such as water or saline, can be directed through the visualization catheter 120 while it is gently pushed through the fallopian tube 220. This allows for selective visualization of the tube 220. Once the catheter 120 has passed through the tube 120, water or other fluid can be directed into the patient's pelvic cavity (FIG. 3). The patient's hips can be lifted slightly to move the bowel out of the pelvis, and additional fluid can be injected to help move the bowel out of the way.

The surgeon or other suitable medical professional then can then examine the ovaries and pelvis underwater to detect any areas exhibiting symptoms or structures consistent with endometriosis or other disorders. Once this has been completed, some of the fluid can be aspirated back through catheter 120 and sent to cytology for analysis. This approach can therefore be used for ovarian cancer screening and to look for endometrial cells that are an indication for endometriosis. The surgeon can then visualize the other fallopian tube as well in a similar fashion.

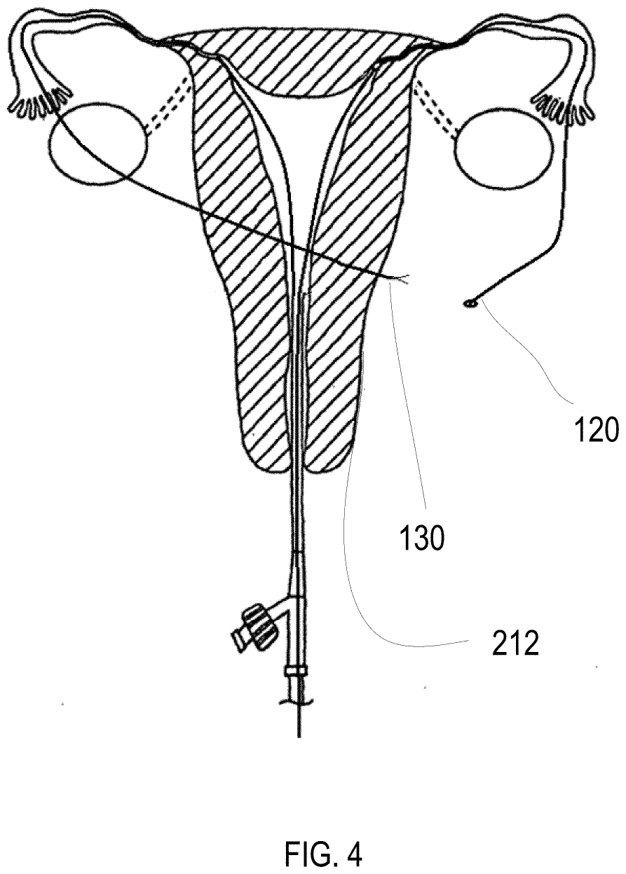

If desired, and as illustrated in FIG. 4, a second catheter 130 slidably disposed within scope 100 (or other catheter) can similarly be caused to traverse the patient's other fallopian tube, exit the tube and be manipulated to another location for purposes, for example, of visualizing the external surface of the uterus, the bowel, the peritoneum, and the like. Devices 120, 130 can be used in concert to visualize a tissue structure and, if desired, take a sample of the tissue sample for analysis. Moreover, one of the scopes 120, 130 can be configured to discharge a laser pulse at a target within the patient's reproductive system to treat endometriosis such as by ablating tissue structures. It will be appreciated that the distal ends of the scopes or portions thereof 100, 120, 130 can be provided with atraumatic distal tips, such as rounded lens elements that in turn can be provided with a lubricious surface or the like.

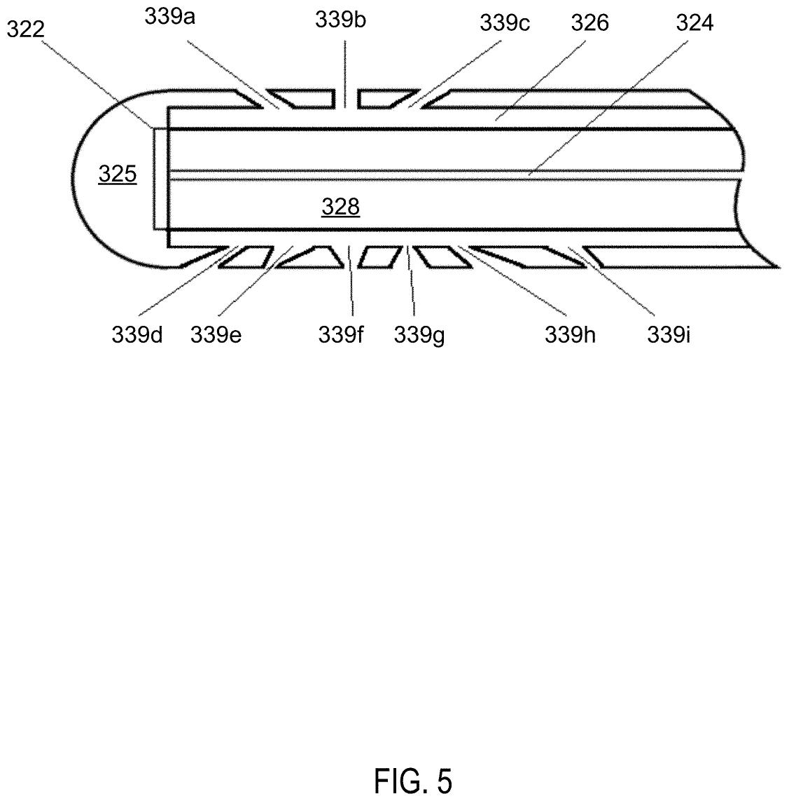

The scopes described herein can be provided with various passageways to permit the passage of fluids therethrough for purposes of irrigation for purposes of lubrication, the cleaning of tools or equipment, for example, aspiration of fluids or cells, and/or the delivery of a beneficial agent, such as a fluid such as saline, or one or more medicaments, such as one or more pharmacological compounds and the like, and/or delivery of light or other radiation, such as a laser beam, delivering electrical energy to tissue to be treated, and the like. For purposes of illustration, FIG. 5 illustrates a distal end portion of a scope as set forth herein, whether it be slidably disposed in another device, such as scope 120, or a scope that is integrated with a medical instrument or that is a standalone instrument.

As illustrated in FIG. 5, the distal end portion of the scope can include an imaging circuit that includes a CMOS chip 322, for example, coupled to transmission circuitry 324. The distal end portion of the scope can include a central core portion or rod or tube 328 that is surrounded by a sleeve 325 that can in turn include a contoured tip that forms a lens over the chip 322. The chip 322 can be mounted on a distal end of the core portion or rod or tube, and it may include a lumen to permit passage of the transmission circuitry 324 therethrough. An inner surface of the sleeve 325 and an outer surface of the core 328 can cooperate to define an annular cavity 326. Annular cavity 326 can be in fluid communication with a source of pressurized fluid, such as a syringe, outside of the patient. One or more jets or passageways can be defined through the wall of the sleeve 325 that can direct pressurized fluid in a predetermined direction. For example, the jet can include a geometry that directs the pressurized fluid distally 329a, wherein the jet has a generally constant cross section along its length. Jets 339d, 339e present variations wherein the jet directs pressurized fluid distally, but along a diverging (diffuser) or converging (nozzle) flow path, respectively. The jet can have a generally constant cross section along its length and be directed radially outwardly as with jet 339b. Jets 339g, 339f present variations wherein the jet directs pressurized fluid radially outwardly, but along a diverging (diffuser) or converging (nozzle) flow path, respectively. The jet 339c can have a generally constant cross section along its length and be directed proximally. Jets 339h, 339i present variations wherein the jet directs pressurized fluid proximally, but along a diverging (diffuser) or converging (nozzle) flow path, respectively.

The device set forth in FIGS. 1-4 can be a single piece device that includes a scope that can be provided with fluid delivery channels, as set forth herein. In some implementations, the outer diameter of the device can be between about 1.5 and 2.0 millimeters, or any increment therebetween of a tenth of a millimeter. Any scope herein can be provided with a hydrophobic coating along all or a part of its length, such as by shrinking a thin walled hydrophobic (e.g., PTFE, PVDF or other fluoropolymer) sleeve around its periphery. If desired, any scope herein can be coated with a hydrophilic coating along all or part of its length (e.g., polyvinylpyrrolidone “PVP” or other suitable material). If desired, any scope herein can be provided with a coating of a lubricant along all or a portion of its length, such as silicone oil and the like. If desired, any scope herein can be provided with a steering capability, such as by way of one or more steering wires.

In accordance with further aspects, any scope set forth herein can be provided with an outer surface that has an enhanced or otherwise increased surface area that is configured to collect a tissue sample by brushing across the tissue. This can be used to obtain a tissue sample, for example, in the ovaries, fallopian tubes, cervix, uterus or abdominal cavity, for example. The enhanced surface area can be provided, for example, by providing an external sheath to the scope that includes an external layer formed, for example, of a braided hollow woven suture material. The material of the suture can in turn include a coating, if desired, to enhance its lubricity and/or its ability to collect a tissue sample. In use, this outer surface can be sent to cytology with a fluid sample from the patient. The suturing material can include small bristles, for example, to enhance their tissue collection capability.

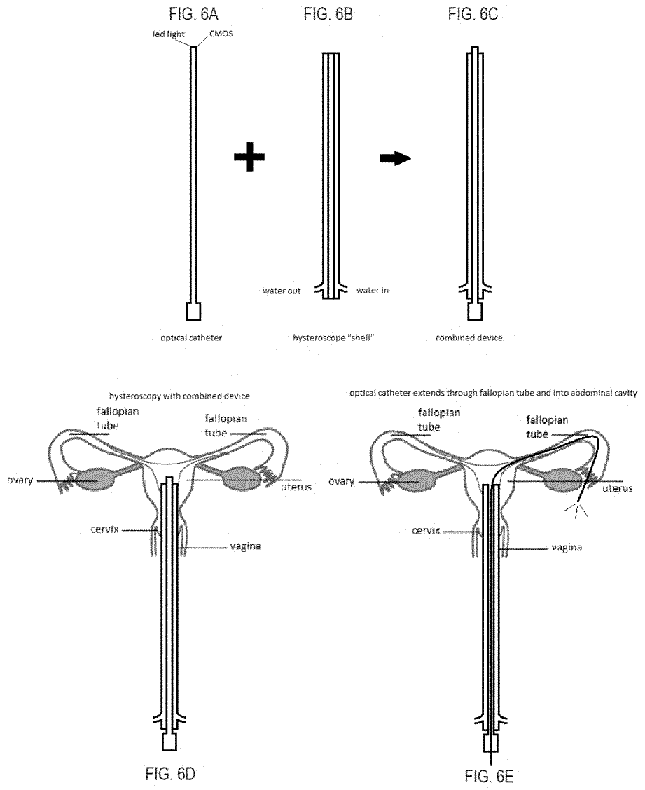

FIGS. 6A-6E illustrate a further embodiment in accordance with the present disclosure. The dimensions illustrated are meant only as examples and are not intended to be limiting. For example, a visualization scope/optical catheter as disclosed herein can be provided having a size of 5 French (1.6 mm diameter) as illustrated in FIG. 6A having illumination and visualization capability. This device can be slidably disposed within a sleeve of a second device (FIG. 6B) (e.g., 3.5 mm in diameter) that acts as a hysteroscope that can, for example flush liquid and aspirate a sample, the combined device being illustrated in FIG. 6C. This device can be used to perform a hysteroscopy as illustrated in FIGS. 6D and 6E, wherein the optical scope can be advanced along the fallopian tube, and the distal end of the hysteroscope can remain in the uterus.

Thus, the disclosed methods and devices permit a complete examination of the uterus, fallopian tubes, ovaries and pelvis in the office. No general anesthesia is needed. To Applicant's knowledge, this is the first ever in office screening tool for endometriosis, first ever visual screening tool for ovarian cancer, and first ever complete in office visualization tool for the entire gynecologic reproductive system. Thus, a patient can be examined over time in the office in an outpatient procedure, for example, to see how the patient is responding to a regimen of treatment. Direct visualization can help reduce or even eliminate the need for exploratory surgery, thereby reducing the cost of care significantly, and the system can be used to deliver one or more beneficial agents (e.g., medicaments, pharmacological compounds and the like) to a target location in a patient's anatomy as set forth above.

It will be appreciated that the present disclosure also includes embodiments of methods and devices that use the above described inner visualization scope (e.g., 5 French diameter) in combination with a standard hysteroscope, wherein the standard hysteroscope defines a (e.g., 5 French diameter) channel therethrough that can be used to receive the visualization scope.

In further implementations, the systems and methods can include a further irrigation and aspiration catheter (e.g, 5 Fr diameter) that is used in the same procedure as the visualization scope to provide enhanced irrigation and suction. Such a further catheter can be used for injecting fluid into the fallopian tubes and into the peritoneal cavity and then to suction out water from the peritoneal cavity. For example, this further irrigation catheter can be introduced into the patient and into one of the patient's Fallopian tubes by inserting the catheter into a patent inside of a standard hysteroscope having a (e.g., 5 Fr) channel defined therethrough. Fluid (e.g., water, saline) can then be injected into the patient by way of the irrigation catheter. This irrigation catheter can then be removed from the outer catheter, and a visualization catheter as set forth herein can be inserted through the lumen of the outer catheter, through the Fallopian tube and into the peritoneal cavity, for example, to inspect organs or other tissue structures. Once visual inspection is complete, the visualization catheter can be withdrawn, and the irrigation catheter can be reintroduced through the outer catheter, for example, into the Fallopian tube and peritoneal cavity to aspirate or otherwise collect fluid and tissue for analysis. The visualization catheter and/or the irrigation catheter can additionally be provided with a surface configured to collect tissue samples, such as an uneven surface with ridges or bumps and depressions, structures resembling cilia or hairs on the surface, or a combination of these features. The catheter collecting the cellular specimens can then be sent to a cytology lab for analysis. The uneven collection surface can be formed, for example, by way of a surface treatment, such as embossing the surface of the catheter, by cutting depressions into it using a laser, and the like. The embossing or laser cutting of the surface (or of a portion of the inner catheter) can also act to decrease bending stiffness of the catheter, but seek to maintain hoop strength of the catheter so that the lumen of the inner catheter, if provided, does not collapse when under fluid suction, such as when a sample is being aspirated. For example an embossing or laser ablation process can form partial and/or full circumferential channels about the inner catheter that enhance bending and maintain hoop strength and also form depressions in the surface for collecting tissue samples. The resulting pattern can resemble, for example, a screw thread or helical pattern, or a pattern of indentations, as desired.

In further accordance with the disclosure, methods and devices are provided to access the recto-uterine pouch, also known by various other names (e.g., cul de sac), is the extension of the peritoneal cavity between the rectum and the posterior wall of the uterus in the female human body. It is the deepest point of the peritoneal cavity. In accordance with the present disclosure, systems and methods are provided to access and extract fluid or tissue from this anatomy. This can be particularly useful for patients with blocked fallopian tubes, or even after a hysterectomy. In an illustrative implementation, a surgeon or other suitable medical personnel can insert a needle, such as a 2 mm needle (12 gauge) having a 1.6 mm (5 French) inner diameter through the posterior of the cul de sac. The needle may be introduced by way of the vagina and puncture through the posterior cul de sac, for example, under a visualization technique such as ultrasound or the like. The visualization scope, or inner scope referenced elsewhere herein, and/or the irrigation catheter discussed elsewhere herein can then be introduced to introduce and/or collect fluid or tissue samples, and to observe tissue structures in the cul de sac and/or to deliver a beneficial agent, such as a fluid such as saline, or one or more medicaments, such as one or more pharmacological compounds and the like, and/or delivery of light or other radiation, such as a laser beam, delivering electrical energy to tissue to be treated, and the like.

The methods and devices provided by the present disclosure, as described above and shown in the drawings, provide for methods and systems for medical diagnosis and treatment with superior properties as described herein. It will be apparent to those skilled in the art that various modifications and variations can be made in the embodiments of the present disclosure described herein without departing from the spirit or scope of the disclosure. Thus, it is intended that the present disclosure include modifications and variations that are within the scope of the appended claims and their equivalents.

1. A diagnostic method for evaluating an intrapelvic condition, comprising:
introducing a visualization scope having a proximal end and a distal end into a patient's uterus by way of the vagina and cervix;
advancing the distal end of the visualization scope into one of the fallopian tubes from the uterus; and
advancing the distal end of the visualization scope out of said fallopian tube into an abdominal cavity of the patient.

2. The method of claim 1, wherein the visualization scope is directed from outside the patient through the vagina, uterus, and fallopian tube into the abdominal cavity without puncturing a tissue structure.

3. The method of claim 1, further comprising:
directing liquid through the visualization scope when the distal end of the visualization scope is in said fallopian tube to distend said fallopian tube; and
examining at least one structure in said distended fallopian tube for an abnormality.

4. The method of claim 1, further comprising advancing the distal end of the visualization scope into a pelvic region of said abdominal cavity.

5. The method of claim 1, further comprising directing liquid through the visualization scope when the distal end of the visualization scope is in a pelvic regain of said abdominal cavity and examining at least one anatomical structure within the pelvic region of the abdominal cavity for at least one abnormality.

6. The method of claim 1, further comprising detecting at least one abnormality associated with endometriosis using the visualization scope.

7. The method of claim 1, further comprising using the visualization scope as a screening tool to detect at least one abnormality associated with cancer, and further wherein the method includes examining at least one anatomical structure selected from the group consisting of (i) a surface of the patient's uterus, (ii) at least one of the patient's ovaries, (iii) the patient's bowels, (iv) at least one of the patient's fallopian tubes, and (v) the patient's peritoneum.

8. The method of claim 1, further comprising:
aspirating a fluid sample from said abdominal cavity; and
performing at least one testing procedure on the fluid sample in order to detect at least one abnormality.

9. The method of claim 8, wherein the at least one testing procedure is configured to detect at least one of (i) cancerous tissue, and (ii) endometriosis.

10. The method of claim 1, wherein the visualization scope includes an electronic photodetector chip disposed proximate to the distal end of the visualization scope for receiving incoming light and a light emitting device proximate to the distal end of the visualization scope, and the method further comprises directing light onto a tissue structure and observing the tissue structure using the electronic photodetector chip.

11. The method of claim 1, further comprising tilting the pelvis of the patient in order to move the bowels out of the way of the visualization scope.

12. The method of claim 1, further comprising directing liquid into a pelvic cavity by way of the visualization scope.

13. The method of claim 1, further comprising conducting a diagnostic or treatment regimen using the visualization scope, and repeating the method a plurality times over a plurality of examinations in order to track progress of a treatment regimen of the patient.

14. The method of claim 1, further comprising directing a therapeutic agent through the visualization scope to treat tissue inside of the patient.

15. The method of claim 1, wherein the visualization scope includes an uneven outer surface configured to collect a tissue sample from a patient as the visualization scope passes over tissue of a patient.

16. The scope of claim 15, wherein the uneven outer surface includes a plurality of hair-like elements.

17. A method for evaluating an intrapelvic condition, comprising:
introducing a visualization scope having a proximal end and a distal end into a patient's cul de sac; and
performing at least one of a diagnostic or therapeutic procedure inside the patient's cul de sac.

18. The method of claim 17, further comprising introducing a needle through the vagina and into the cul de sac to define a passageway through which the visualization scope can pass.

19. The method of claim 17, wherein the diagnostic procedure includes at least one of aspirating fluid and obtaining a tissue sample.

20. The method of claim 17, wherein the therapeutic procedure includes delivering a beneficial agent to tissue in the cul de sac.
